(19) United States
(12) Patent Application Publication
(10) Pub. No.: US 2010/0240900 A1
(43) Pub. Date: Sep. 23, 2010

(54) DISPERSIBLE CARBON NANOSPHERES AND METHODS FOR MAKING SAME

(75) Inventors: Cheng Zhang, Pennington (CN); Gaurang Bhargava, Plainsboro, NJ (US); Bing Zhou, Cranbury, NJ (US)

Correspondence Address: Workman Nydegger;1000 Eagle Gate Tower, 60 East South Temple, Salt Lake City, UT, 84111, US

(73) Assignee: HEADWATERS TECHNOLOGY INNOVATION, LLC, Lawrenceville, NJ (US)

(21) Appl. No.: 12/409,168

(22) Filed: Mar. 23, 2009

(51) Int. Cl. C07D 213/26; C07D 213/30; C07D 213/06

(52) U.S. Cl. 546/330; 546/347; 546/348; 546/344; 546/340; 977/773; 977/788

(57) ABSTRACT

The carbon nanomaterials and methods relate to methods for causing carbon nanospheres to be readily dispersible in a material. The carbon nanospheres are rendered dispersible using a cationic surfactant. The surfactant includes one or more cationic group that can bond to the surface of the carbon nanospheres, without detrimentally affecting the unique properties of carbon nanospheres. The dispersible carbon nanospheres can be dried (i.e., solvent is driven off) while maintaining their dispersibility in solvents and other materials.

### BACKGROUND OF THE INVENTION

1. The Field of the Invention

The present invention relates generally to the manufacture of dispersible carbon nanospheres.

2. The Related Technology

Carbon materials have been used in various fields for a variety of applications. Examples of current uses of carbon materials include pigments, fillers, catalyst supports, and fuel cell electrodes, among others. Pyrolysis of organic compounds is a known method for preparing carbon materials. For example, carbon materials can be produced by pyrolyzing resorcinol-formaldehyde gel at temperatures above 600° C.

Most carbon materials obtained by pyrolysis of organic compounds at temperatures between 600-1400° C. tend to be amorphous or have a disordered structure. Obtaining highly crystalline or graphitic carbon materials can be very advantageous because of the unique properties exhibited by graphite. For example, graphitic materials can be thermally and electrically conductive.

### BRIEF SUMMARY OF THE INVENTION

The carbon nanomaterials and methods described herein relate to methods for making carbon nanospheres readily dispersible in a material. The carbon nanospheres are rendered dispersible using a cationic surfactant. The selection of the surfactant is critical to achieving the desired dispersibility of the carbon nanospheres. The surfactant includes a cationic group that can bond to the surface of the carbon nanospheres without detrimentally affecting the unique properties of carbon nanospheres. In addition, the surfactants of the present invention are stable independent from the solvent.

To obtain the dispersed carbon nanospheres, a cationic surfactant is mixed with the carbon nanospheres under conditions suitable for bonding the cationic surfactant to the surface of the carbon nanospheres. Two types of cationic surfactants that can be independently used to yield superior carbon nanospheres include aromatic cationic surfactants and dimeric cationic surfactants.

Typically, the cationic surfactant is dissolved in a solvent and mixed with the carbon nanospheres. The cationic group of the cationic surfactant ionically bonds to the surface of the carbon nanospheres. The solvent can be removed and the ionically bonded surfactant remains bonded to the surface of the carbon nanospheres. Advantageously, the dispersible carbon nanospheres can be dried (i.e., solvent driven off) while maintaining their dispersibility in solvents and other materials.

In one embodiment, the mixing of the cationic surfactant and the carbon nanospheres can be carried out using sonication. The sonication treatment, in combination with the cationic surfactant, can be much more effective than either sonication or use of the cationic surfactant alone relative to dispersing the carbon nanospheres in a material.

As mentioned, in some embodiments, the surfactant can include an aromatic ring. The cationic group can be included in the aromatic ring or can be adjacently attached to the ring. According to one embodiment, the cationic group is preferably in or part of the aromatic ring. Cationic surfactants that include an aromatic ring have been found to give surprisingly high conductivity and dispersibility. An example of a cationic surfactant with a cationic group in an aromatic ring is hexadecylpyridine, 1-(2-hydroxyethyl)pyridinium chloride, 1-(3-Cyanopropyl)pyridinium chloride, 1-Butyl-4-methylpyridinium chloride, 1-(1-(ethoxycarbonyl)tridecyl)pyridinium bromide.

In an alternative embodiment, the cationic surfactant is a dimeric cationic surfactant, which has at least two cationic groups separated by a carbon chain. These cationic surfactants have been found to increase the density of carbon nanospheres without substantially diminishing conductivity of the carbon nanospheres and can be dried and reconstituted in a solvent. In one embodiment, the density of the dispersed carbon nanospheres is at least about 0.4 g/ml. Examples of dimeric cationic surfactants that can be used in the present invention include, but are not limited to, butane-1,4 bis(dodecyldimethyl ammonium chloride),

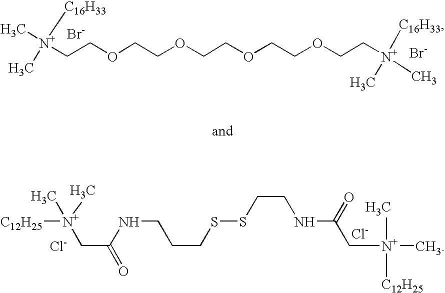

The carbon nanospheres dispersed using the methods of the invention have been found to retain the beneficial structure, shape, and graphitic nature of the undispersed carbon nanospheres. The dispersed carbon nanospheres are highly graphitic, which is advantageous for providing strength, electrical conductivity, thermal conductivity and other desired properties.

These and other advantages and features of the present invention will become more fully apparent from the following description and appended claims as set forth below.

### DETAILED DESCRIPTION OF EXAMPLE EMBODIMENTS

### I. INTRODUCTION AND DEFINITIONS

The carbon nanomaterials and methods described herein relate to methods for making carbon nanospheres readily dispersible in a solvent or solid material. The cationic surfactants used to render the carbon nanospheres dispersible are selected to modify the surface of the carbon nanospheres without detrimentally affecting the unique properties of carbon nanospheres. Advantageously, the dispersible carbon nanospheres can be dried (i.e., solvent is driven off) while still maintaining their dispersibility in solvents and other materials.

For purposes of the present invention, the average particle size of the carbon nanomaterial is determined using dynamic light scattering and corresponds to a peak of the light scattering spectral data. Where more than one significant peak is observed, the average diameter shall mean the weighted average according to the % intensity for the two or more peaks, unless otherwise indicated.

### II. COMPONENTS USED TO MANUFACTURE DISPERSED CARBON NANOSPHERES

A. Carbon Nanomaterials Containing Carbon Nanospheres

The carbon nanospheres used in the method of the invention are multi-walled, hollow, graphitic structures with an average diameter in a range from about 10 nm to about 200 nm, preferably about 20 nm to about 100 nm. The multiple walls form a closed structure with a hollow center.

Typically, the individual carbon nanospheres have an aspect ratio of less than about 3:1 (i.e., width to height is less than 3:1), preferably less than about 2:1, more preferably less than about 1.75:1, and most preferably less than about 1.5:1. In one embodiment, the carbon nanospheres have an irregular surface. The carbon nanospheres are highly graphitic, which gives the carbon nanomaterial excellent electrical and thermal conductivity.

Typically, the thickness of the carbon nanosphere wall is between about 1 nm and 20 nm. However, thicker and thinner walls can be made if desired. The thickness of the nanostructure wall is measured from the inside diameter of the wall to the outside diameter of the wall. In one embodiment, the carbon nanostructures have walls of between about 2 and about 100 graphite layers, preferably between about 5 and 50 graphite layers, and more preferably between about 5 and 20 graphite layers. The number of graphitic layers can be varied by varying the thickness of the carbon nanostructure wall as discussed below in relation to methods for manufacturing carbon nanospheres. The advantage of making a thicker wall is greater structural integrity. The advantage of making a thinner wall is greater surface area and nanoporosity.

The spheroidal shape and multi-walled nature of the carbon nanospheres also provides strength that makes the carbon nanospheres less likely to be crushed or broken into undesired shapes or non-shaped graphite. Maintaining the shape of the carbon nanospheres can be important for maintaining performance characteristics over time. The multi-walled nature of the nanospheres also allows the surface to be functionalized while maintaining the beneficial thermal and electrical conductivity via the interior graphite layers. The hollow center gives the nanomaterial a relatively lower density and higher porosity. In one embodiment, the surface area is in a range from about 100 m²/g to about 400 m²/g, preferably about 125 m²/g to about 300 m²/g, and more preferably about 150 m²/g to about 250 m²/g.

In one embodiment, the carbon nanospheres can be one of several components of a carbon nanomaterial. Higher percentages of carbon nanospheres are typically preferred such that the carbon nanomaterial can benefit from the unique properties of the carbon nanospheres. In one embodiment, the nanospheres are at least about 10 wt % of the carbon nanomaterial, preferably at least about 50 wt %, more preferably about 75 wt %, even more preferably at least about 90 wt %, and most preferably at least about 98 wt %. The portion of the carbon nanomaterial that is not carbon nanospheres is preferably a graphitic material such as graphite sheets or other graphitic nanostructures. The carbon nanomaterials can include non-graphitic amorphous carbon. However, it is typically advantageous to minimize the percentage of non-graphitic amorphous carbon (e.g., by removing it during purification and/or by converting non-graphitic amorphous carbon to graphite during additional heat treatment steps).

B. Cationic Surfactants

The cationic surfactants used in the methods disclosed herein include a cationic group that can bond with the surface of the carbon nanospheres. The carbon nanospheres are highly graphitic and therefore are electron rich. The cationic group has a positive charge that can bond through inductive charge interactions. The cationic surfactant is chosen such that the ionic bonding allows the dispersible carbon nanospheres to be dried, washed, reconstituted, and/or mixed with other materials without removing the cationic surfactant.

In addition to the cationic group, the cationic surfactants can have functional groups that give the cationic surfactant desired properties. For example, in a preferred embodiment, the cationic surfactant includes one or more hydrocarbon chains that increase the density of the dispersible carbon nanospheres. In a preferred embodiment, the cationic surfactant includes one or more carbon side chains having between 4 and 18 carbons, more preferably between about 8 and 12 carbons. Cationic surfactants that include hydrocarbon carbon chains can be made hydrophobic and be dispersible in hydrophobic materials. Although the cationic surfactants include a cationic group, the dispersible carbon nanospheres can be highly dispersible in hydrophobic materials since the charge on the cationic group is bonded with the electron rich carbon nanosphere surface, which permits the hydrophobic portion of the cationic surfactant to have a dominate role in solvent and/or material interactions.

In some embodiments, the surfactant can include an aromatic ring. The aromatic ring can contribute to bonding to the surface of the carbon nanospheres through pi-pi stacking interactions. Cationic surfactants that include an aromatic ring have been found to give surprisingly high conductivity and dispersibility.

The cationic group can be included in the aromatic ring or can be adjacently attached to the ring. Cationic groups positioned in the ring have been found to significantly increase the conductivity of the carbon nanospheres while improving their dispersibility in solvents and other materials. If the cationic group is not placed in the ring, the cationic group is preferably separated by a single atom (e.g., carbon) or more preferably directly bonded to the aromatic ring.

The cationic surfactants with an aromatic ring can include a hydrophobic side chain attached to the aromatic ring. The hydrophobic side chain preferably includes about 4 to about 18 carbons, more preferably about 8 to about 12 carbons, although other chain lengths can also be used.

Examples of cationic surfactants having an aromatic ring that can be used in the present invention, include but are not limited to, hexadecyl pyridine chloride, hexadecylpyridine, 1-(2-hydroxyethyl)pyridinium chloride, 1-(3-Cyanopropyl)pyridinium chloride, 1-Butyl-4-methylpyridinium chloride, 1-(1-(ethoxycarbonyl)tridecyl)pyridinium bromide.

In another embodiment, the cationic surfactant can include at least two cationic groups separated by a carbon chain (i.e., dimeric cationic surfactants). The dimeric cationic surfactants have two or more amphiphilic domains that each include a cationic group and a hydrophobic group. The amphiphilic domains are joined together through the cationic groups or through the hydrophobic groups. The hydrocarbon chains linking the cationic groups together form a chain of atoms that is at least about 4 to about 18 atoms long, more preferably about 8 to about 12 atoms long.

Examples of suitable dimeric cationic surfactants include butane-1,4 bis(dodecyldimethyl ammonium chloride),

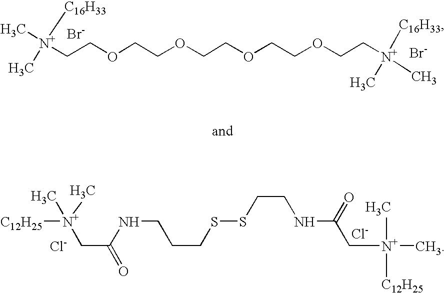

C. Solvents

Various solvents can be used in the methods and compositions described herein. The solvent is typically used to dilute and/or deliver the cationic surfactant to the surface of the carbon nanospheres. Solvents can also be used to wash the dispersible carbon nanospheres to remove unbounded or excess cationic surfactant. A solvent can also be used to suspend the carbon nanospheres before and/or after reacting the cationic surfactant with the carbon nanospheres. Examples of suitable solvents that can be used in the methods described herein include, but are not limited to, water, alcohols, such as but not limited to ethanol and methanol, tetrahydrofuran (THF), DMF, and the like.

### III. METHODS FOR DISPERSING CARBON NANOSPHERES

In the method of the present invention, the carbon nanospheres are made dispersible by reacting the carbon nanospheres with a cationic surfactant. The cationic surfactant is typically dissolved in a suitable solvent and then mixed with the carbon nanospheres and allowed to react.

The surfactant can be included in the reaction mixture in a concentration in a range from about 0.1 weight percent to about 80 weight percent, more preferably about 0.5 weight percent to about to about 20 weight percent, and most preferably about 5 weight percent to about 10 weight percent. The carbon nanomaterial containing the carbon nanospheres is typically included in the mixture in a concentration in a range from about 0.1 weight percent to about 20 weight percent, more preferably about 1 weight percent to about 10 weight percent.

The reaction temperature is typically in a range from about 30° C. to about 100° C., more preferably in a range from about 50° C. to about 80° C. The reaction can be carried out for a period of time in a range from about 1 h to about 48 h, more preferably, 5 h to about 10 h. However, other concentrations, temperatures, and reaction times can also be used depending on the type and configuration of the cationic surfactant, solvent, and carbon nanospheres.

The carbon nanospheres can be dispersed into the solvent using ultrasonication. The use of ultrasonication in combination with manufacturing the solvents has been found to produce highly dispersed nanospheres. Ultrasonication can be carried out using any suitable technique, such as an ultrasonic bath, to vibrate the carbon nanospheres at ultrasonic frequencies. An example of an ultrasonication device suitable for use in dispersing carbon nanospheres is CREST ULTRASONICS TRU-SWEEP™ (68 kHz frequencies and 500 watt).

Ultrasonication is typically carried out for at least 30 min, preferably at least about 1 hour, and more preferably at least about 2 hours. Examples of suitable ranges of time for carrying out ultrasonication of the mixture include about 30 minutes to about 6 hours and preferably about 1 hour to about 3 hours. The ultrasonication step can be carried out at room temperature or other suitable temperatures.

The combination of the solvent, cationic surfactant, and ultrasonication is able to break up and disperse agglomerates of carbon nanospheres. Unexpectedly, agglomerates of carbon nanospheres with an average particle size of 1-5 microns can be dispersed using the inventive methods to yield nanospheres and/or agglomerates of nanospheres with an average particle size of less than about 300 nm, more preferably less than about 200 nm, and most preferably less than about 150 nm as measured using dynamic light scattering.

In addition to the improved dispersion, the dispersed carbon nanospheres also tend to have a relatively narrow distribution of particle sizes. In one embodiment, the width of the particle size distribution is in a range from about 10 nm to about 300 nm.

Carbon nanospheres dispersed according to the methods of the present invention advantageously retain their beneficial properties such as multi-walled, hollow, closed structure, graphitic nature, and original size and shape of the primary structures.

The carbon nanospheres dispersed according to the present invention have been found to be surprisingly stable in dry form or in a solvent. Carbon nanospheres manufactured according to the present invention have been observed to be stable for months at room temperature. In one embodiment of the invention, the carbon nanospheres are stable for at least about one hour, more preferably at least about one day, and most preferably at least about one month.

Carbon nanospheres manufactured using the aromatic cationic surfactants show surprisingly high conductivity. Conductivity substantially increases with the addition of the surfactant. Moreover, the high conductivity is highly reproducible. In one embodiment, dispersed carbon nanospheres that include an aromatic cationic surfactant bonded thereto has a conductivity of at least about 150 S/m, more preferably at least about 200 S/m, even more preferably at least about 250 S/m, and most preferable at least about 300 S/m. In one embodiment, the conductivity is in a range from about 100 S/m to about 1000 S/m, more preferably about 150 S/m to about 500 S/m, and most preferably in a range from about 200 S/m to about 400 S/m. In one embodiment, the conductivity of the dispersible carbon nanospheres manufactured according to the methods of the invention has a conductivity that is at least about 50 S/m greater than the carbon nanomaterial prior to being reacted with the cationic surfactant, more preferably at least about 100 S/m greater.

Carbon nanospheres manufactured using dimeric cationic surfactants have been found to be highly stable in solvents even after drying and have substantially improved densities. In one embodiment, the density of the dispersible carbon nanospheres is greater than about 0.2 g/ml, more preferably greater than about 0.3 g/ml, and most preferably greater than about 0.4 g/ml. In one embodiment, the density of the dispersed carbon nanospheres compared to the undispersed carbon nanospheres is at least 15% greater, more preferably at least about 25% greater, and most preferably at least about 35% greater compared to the carbon nanosphere prior to reaction with the cationic surfactant.

The dispersed carbon nanospheres are particularly advantageous for making composites. Because the carbon nanospheres are readily dispersible, the carbon nanospheres can be mixed with other materials to form composites. The composites of the invention can benefit from the narrow particle size distribution and unique properties of the carbon nanospheres of the invention, including strength, electrical and thermal conductivity, porosity, surface area, etc.

### IV. MANUFACTURING CARBON NANOSPHERES

The carbon nanospheres used in the methods of the present invention can be manufactured using any technique that provides carbon nanospheres having the desired properties described above. In one embodiment, the method for manufacturing carbon nanospheres generally includes (1) forming a precursor mixture that includes a carbon precursor and a plurality of catalytic templating particles, (2) carbonizing the precursor mixture to form an intermediate carbon material including carbon nanostructures, amorphous carbon, and catalytic metal, and (3) purifying the intermediate carbon material by removing at least a portion of the amorphous carbon and optionally at least a portion of the catalytic metal. The following components can be used to carry out the above mentioned steps for manufacturing carbon nanospheres according to the present invention.

A. Components Used to Make Carbon Nanospheres

(1) Carbon Precursor

Any type of carbon material can be used as the carbon precursor of the present invention so long as it can disperse the templating particles and carbonize around the templating particles upon heat treating. Suitable compounds include single and multi-ring aromatic compounds such as benzene and naphthalene derivatives that have polymerizable functional groups. Also included are ring compounds that can form single and multi-ring aromatic compounds upon heating. Functional groups that can participate in polymerization include COOH, C═O, OH, C═C, SO₃, NH₂, SOH, N═C═O, and the like.

The carbon precursor can be a single type of molecule (e.g., a compound that can polymerize with itself), or the carbon precursor can be a combination of two or more different compounds that co-polymerize together. For example, in one embodiment, the carbon precursor can be a resorcinol-formaldehyde gel. In this two compound embodiment, the formaldehyde acts as a cross-linking agent between resorcinol molecules by polymerizing with the hydroxyl groups of the resorcinol molecules.

Other examples of suitable carbon precursors include resorcinol, phenol resin, melamine-formaldehyde gel, poly(furfuryl alcohol), poly(acrylonitrile), sucrose, petroleum pitch, and the like. Other polymerizable benzenes, quinones, and similar compounds can also be used as carbon precursors and are known to those skilled in the art.

In one embodiment, the carbon precursor is a hydrothermally polymerizable organic compound. Suitable organic compounds of this type include citric acid, acrylic acid, benzoic acid, acrylic ester, butadiene, styrene, cinnamic acid, and the like.

(2) Catalytic Templating Nanoparticles

The catalytic templating nanoparticles are used as a template for making the nanostructures. When mixed with the carbon precursor, the templating nanoparticles provide a nucleation site where carbonization and/or polymerization can begin or be enhanced. Because the templating nanoparticles are made from catalytic atoms, the templating particles can advantageously serve as both a nucleating site and as a catalyst for carbonization and/or polymerization.

The catalytic templating particles can be formed in more than one way. As described below, in one embodiment, the templating particles are formed from metal salts that agglomerate to form particles. Optionally, the catalyst atoms can be complexed with a dispersing agent to control formation of the particles. Templating nanoparticles formed using a dispersing agent tend to be more uniform in size and shape compared to templating particles formed without a dispersing agent.

(i) Catalyst Atoms

The catalyst atom used to form the templating nanoparticles can be any material that can cause or promote carbonization and/or polymerization of the carbon precursor. In a preferred embodiment, the catalyst is a transition metal catalyst including, but not limited to, iron, cobalt, or nickel. These transition metal catalysts are particularly useful for catalyzing many of the polymerization and/or carbonization reactions involving the use of the carbon precursors described above.

(ii) Dispersing Agents

Optionally, a dispersing agent can be complexed with the catalyst atoms to control formation of the templating nanoparticles. The dispersing agent is selected to promote the formation of nanocatalyst particles that have a desired stability, size and/or uniformity. Dispersing agents within the scope of the invention include a variety of small organic molecules, polymers and oligomers. The dispersing agent is able to interact and bond with catalyst atoms dissolved or dispersed within an appropriate solvent or carrier through various mechanisms, including ionic bonding, covalent bonding, Van der Waals interaction/bonding, lone pair electron bonding, or hydrogen bonding.

To provide the bonding between the dispersing agent and the catalyst atoms, the dispersing agent includes one or more appropriate functional groups. Preferred dispersing agents include functional groups which have either a charge or one or more lone pairs of electrons that can be used to complex a metal catalyst atom, or which can form other types of bonding such as hydrogen bonding. These functional groups allow the dispersing agent to have a strong binding interaction with the catalyst atoms.

The dispersing agent may be a natural or synthetic compound. In the case where the catalyst atoms are metal and the dispersing agent is an organic compound, the catalyst complex so formed may be an organometallic complex.

In one embodiment, the functional groups of the dispersing agent comprise one or more members selected from the group of a hydroxyl, a carboxyl, a carbonyl, an amine, an amide, a nitrile, a nitrogen with a free lone pair of electrons, an amino acid, a thiol, a sulfonic acid, a sulfonyl halide, or an acyl halide. The dispersing agent can be monofunctional, bifunctional, or polyfunctional.

Examples of suitable monofunctional dispersing agents include alcohols such as ethanol and propanol and carboxylic acids such as formic acid and acetic acid. Useful bifunctional dispersing agents include diacids such as oxalic acid, malic acid, malonic acid, maleic acid, succinic acid, and the like; dialcohols such as ethylene glycol, propylene glycol, 1,3-propanediol, and the like; hydroxy acids such as glycolic acid, lactic acid, and the like. Useful polyfunctional dispersing agents include sugars such as glucose, polyfunctional carboxylic acids such as citric acid, pectins, cellulose, and the like. Other useful dispersing agents include ethanolamine, mercaptoethanol, 2-mercaptoacetate, amino acids, such as glycine, and sulfonic acids, such as sulfobenzyl alcohol, sulfobenzoic acid, sulfobenzyl thiol, and sulfobenzyl amine. The dispersing agent may even include an inorganic component (e.g., silicon-based).

Suitable polymers and oligomers within the scope of the invention include, but are not limited to, polyacrylates, polyvinylbenzoates, polyvinyl sulfate, polyvinyl sulfonates including sulfonated styrene, polybisphenol carbonates, polybenzimidizoles, polypyridine, sulfonated polyethylene terephthalate. Other suitable polymers include polyvinyl alcohol, polyethylene glycol, polypropylene glycol, and the like.

In addition to the characteristics of the dispersing agent, it can also be advantageous to control the molar ratio of dispersing agent to the catalyst atoms in a catalyst suspension. A more useful measurement is the molar ratio between dispersing agent functional groups and catalyst atoms. For example, in the case of a divalent metal ion two molar equivalents of a monovalent functional group would be necessary to provide the theoretical stoichiometric ratio. In a preferred embodiment, the molar ratio of dispersing agent functional groups to catalyst atoms is preferably in a range of about 0.01:1 to about 100:1, more preferably in a range of about 0.05:1 to about 50:1, and most preferably in a range of about 0.1:1 to 20:1.

The dispersing agents of the present invention allow for the formation of very small and uniform nanoparticles. In general, the nanocatalyst particles formed in the presence of the dispersing agent are less than 1 micron in size. Preferably the nanoparticles are less than about 100 nm, more preferably less than about 50 nm, and most preferably less than about 20 nm.

During pyrolysis of the carbon precursor, the dispersing agent can inhibit agglomeration and deactivation of the catalyst particles. This ability to inhibit deactivation can increase the temperature at which the nanocatalysts can perform and/or increase the useful life of the nanocatalyst in the extreme conditions of pyrolysis. Even if including the dispersing agent only preserves catalytic activity for a few additional milliseconds, or even microseconds, the increased duration of the nanocatalyst can be very beneficial at high temperatures, given the dynamics of carbonization.

(iii) Solvents and Other Additives

A solvent can optionally be used to prepare the catalyst atoms for mixing with the dispersing agent and/or the carbon precursor. The liquid medium in which the catalytic templating nanoparticles are prepared may contain various solvents, including water and organic solvents. Solvents participate in particle formation by providing a liquid medium for the interaction of catalyst atoms and dispersing agent. In some cases, the solvent may act as a secondary dispersing agent in combination with a primary dispersing agent that is not acting as a solvent. In one embodiment, the solvent also allows the nanoparticles to form a suspension. Suitable solvents include water, methanol, ethanol, n-propanol, isopropyl alcohol, acetonitrile, acetone, tetrahydrofuran, ethylene glycol, dimethylformamide, dimethylsulfoxide, methylene chloride, and the like, including mixtures thereof.

The catalyst composition can also include additives to assist in the formation of the nanocatalyst particles. For example, mineral acids and basic compounds can be added, preferably in small quantities (e.g., less than 5 wt %). Examples of mineral acids that can be used include hydrochloric acid, nitric acid, sulfuric acid, phosphoric acid, and the like. Examples of basic compounds include sodium hydroxide, potassium hydroxide, calcium hydroxide, ammonium hydroxide, and similar compounds.

It is also possible to add solid materials to assist in nanoparticle formation. For example, ion exchange resins may be added to the solution during catalyst formation. Ion exchange resins can be substituted for the acids or bases mentioned above. Solid materials can be easy separated from the final iron catalyst solution or suspension using simple techniques such as centrifugation and filtration.

(3) Reagents for Purifying Intermediate Carbon Materials

Various reagents can be used to remove amorphous carbon and/or the catalytic metals from the carbon nanostructures, thereby purifying the intermediate material. The purification can be carried out using any reagent or combination of reagents capable of selectively removing amorphous carbon (or optionally catalytic metal) while leaving graphitic material.

Reagents for removing amorphous carbon include oxidizing acids, oxidizing agents, and mixtures of these. An example of a mixture suitable for removing amorphous carbon includes sulfuric acid, KMnO₄, H₂O₂, 5M or greater HNO₃, and aqua regia.

The catalytic metal can be removed using any reagent that can selectively dissolve the particular metal used as catalyst without significantly destroying the carbon nanostructures, which are graphitic. Nitric acid is an example of a reagent suitable for dissolving many base transition metals such as, but not limited to, iron, cobalt, and nickel. Other examples of suitable reagents include hydrogen fluoride, hydrochloric acid, and sodium hydroxide. If desired, additional heat treatment steps can be carried out on the intermediate carbon to convert all or some of the remaining amorphous carbon to graphite. The subsequent heat treatment can be carried out at a temperature above about 250° C., more preferably above about 500° C.

B. Process for Making Carbon Nanospheres

The carbon nanostructures of the present invention can be manufactured using all or a portion of the following steps: (i) forming a precursor mixture that includes a carbon precursor and a plurality of templating nanoparticles, (ii) allowing or causing the carbon precursor to polymerize around the catalytic templating nanoparticles, (iii) carbonizing the precursor mixture to form an intermediate carbon material that includes a plurality of nanostructures (e.g., carbon nanospheres), amorphous carbon, and catalytic metal, and (iv) purifying the intermediate carbon material by removing at least a portion of the amorphous carbon and optionally a portion of the catalytic metal. The purification step can also include removing oxygen containing functional groups generated during the removal of amorphous carbon or adding additional oxygen-containing functional groups to impart greater hydrophilicity to the carbon nanospheres.

(1) Forming a Precursor Mixture

The precursor mixture is formed by selecting a carbon precursor and dispersing a plurality of catalytic templating nanoparticles in the carbon precursor.

The catalytic templating nanoparticles, which are dispersed in the carbon precursor, can be provided in several different ways. The templating nanoparticles can be formed in the carbon precursor (i.e., in-situ) or formed in a separate reaction mixture and then mixed with the carbon precursor. In some cases, particle formation may partially occur in a separate reaction and then be completed as the templating particles are mixed and/or heated in the carbon precursor (e.g., at the onset of a precursor polymerization step). The templating nanoparticles can also be formed using a dispersing agent that controls one or more aspects of particle formation or the templating nanoparticles can be made from metal salts.

In one embodiment, the templating nanoparticles are formed in the carbon precursor from a metal salt. In this embodiment, the templating nanoparticles are formed by selecting one or more catalyst metal salts that can be mixed with the carbon precursor. The metal salts are mixed with the carbon precursor and then allowed or caused to form nanoparticles in-situ.

In a more preferred embodiment, the templating particles are formed (in-situ or ex-situ) using a dispersing agent to control particle formation. In this embodiment, one or more types of catalyst atoms and one or more types of dispersing agents are selected. Next, the catalyst atoms (e.g., in the form of a ground state metal or metal salt) and dispersing agent (e.g., in the form of a carboxylic acid or its salt) are reacted or combined together to form catalyst complexes. The catalyst complexes are generally formed by first dissolving the catalyst atoms and dispersing agent in an appropriate solvent and then allowing the catalyst atoms to bond with the dispersing agent molecules. The various components may be combined or mixed in any sequence or combination. In addition, a subset of the components can be premixed prior to addition of other components, or all components may be simultaneously combined.

In an embodiment of the invention, the components for the templating nanoparticles are allowed or caused to form nanoparticles by mixing the components for a period of about 1 hour to about 14 days. This mixing is typically conducted at temperatures ranging from about 0° C. to about 200° C. In one embodiment, the temperature does not exceed 100° C. Particle formation can also be induced using a reagent. For example, in some cases formation of particles or intermediate particles can be caused by bubbling hydrogen through the solution of catalyst complexes.

The templating nanoparticles of the present invention are capable of catalyzing polymerization and/or carbonization of the carbon precursor. The concentration of catalytic templating nanoparticles in the carbon precursor is typically selected to maximize the number of carbon nanostructures formed. The amount of catalytic templating particles can vary depending on the type of carbon precursor being used. In an example embodiment the molar ratio of carbon precursor to catalyst atoms is about 0.1:1 to about 100:1, more preferably about 1:1 to about 30:1.

(2) Polymerizing the Precursor Mixture

The precursor mixture is typically allowed to cure for sufficient time such that a plurality of intermediate carbon nanostructures are formed around the templating nanoparticles. Because the templating nanoparticles are catalytically active, the templating nanoparticles can preferentially accelerate and/or initiate polymerization of the carbon precursor near the surface of the templating particles.

The time needed to form intermediate nanostructures depends on the temperature, the type and concentration of the catalyst material, the pH of the solution, and the type of carbon precursor being used. During polymerization, the intermediate carbon nanostructures can be individual organic structures or an association of nanostructures that break apart during carbonization and/or removal of amorphous carbon.

Ammonia added to adjust the pH can also effect polymerization by increasing the rate of polymerization and by increasing the amount of cross linking that occurs between precursor molecules.

For hydrothermally polymerizable carbon precursors, polymerization typically occurs at elevated temperatures. In a preferred embodiment, the carbon precursor is heated to a temperature of about 0° C. to about 200° C., and more preferably between about 25° C. to about 120° C.

An example of a suitable condition for polymerization of resorcinol-formaldehyde gel (e.g., with iron particles and a solution pH of 1-14) is a solution temperature between about 0° C. and about 90° C. and a cure time of less than 1 hour to about 72 hours. Those skilled in the art can readily determine the conditions necessary to cure other carbon precursors under the same or different parameters.

In one embodiment the polymerization is not allowed to continue to completion. Terminating the curing process before the entire solution is polymerized can help to form a plurality of intermediate nanostructures that will result in individual nanostructures, rather than a single mass of carbonized material. However, the present invention includes embodiments where the carbon precursor forms a plurality of intermediate carbon nanostructures that are linked or partially linked to one another. In this embodiment, individual nanostructures are formed during carbonization and/or during the removal of amorphous carbon.

Forming intermediate carbon nanostructures from the dispersion of templating nanoparticles causes formation of a plurality of intermediate carbon nanostructures having unique shapes and sizes. Ultimately, the properties of the nanostructure can depend at least in part on the shape and size of the intermediate carbon nanostructure. Because of the unique shapes and sizes of the intermediate carbon nanostructures, the final nanostructures can have beneficial properties such as high surface area and high porosity, among others.

(3) Carbonizing the Precursor Mixture

The precursor mixture is carbonized by heating to form an intermediate carbon material that includes a plurality of carbon nanostructures, amorphous carbon, and catalyst metal. The precursor mixture can be carbonized by heating the mixture to a temperature between about 500° C. and about 2500° C. During the heating process, atoms such as oxygen and nitrogen are volatilized or otherwise removed from the intermediate nanostructures (or the carbon around the templating nanoparticles) and the carbon atoms are rearranged or coalesced to form a carbon-based structure.

The carbonizing step typically produces a graphite based nanostructure. The graphite based nanostructure has carbon atoms arranged in structured sheets of sp² hybridized carbon atoms. The graphitic layers can provide unique and beneficial properties, such as electrical conduction and structural strength and/or rigidity.

(4) Purifying the Intermediate Carbon Material

The intermediate carbon material is purified by removing at least a portion of non-graphitic amorphous carbon. This purification step increases the weight percent of carbon nanostructures in the intermediate carbon material.

The amorphous carbon is typically removed by oxidizing the carbon. The oxidizing agents used to remove the amorphous carbon are selective to oxidation of the bonds found in non-graphitic amorphous carbon but are less reactive to the pi bonds of the graphitic carbon nanostructures. The amorphous carbon can be removed by applying the oxidative agents or mixtures in one or more successive purification steps.

Optionally substantially all or a portion of the catalytic metals can be removed. Whether the catalytic metal is removed and the purity to which the catalytic metal is removed will depend on the desired amount of metal in the final product.

Typically, the templating nanoparticles are removed using acids or bases such as nitric acid, hydrogen fluoride, or sodium hydroxide. The method of removing the templating nanoparticles or amorphous carbon depends on the type of templating nanoparticle or catalyst atoms in the composite. Catalyst atoms or particles (e.g., iron particles or atoms) can typically be removed by refluxing the composite nanostructures in 5.0 M nitric acid solution for about 3-6 hours.

Any removal process can be used to remove the templating nanoparticles and/or amorphous carbon so long as the removal process does not completely destroy the carbon nanostructures. In some cases it may even be beneficial to at least partially remove some of the carbonaceous material from the intermediate nanostructure during the purification process.

During the purification process, the oxidizing agents and acids have a tendency to introduce hydronium groups and oxygenated groups such as, but not limited to, carboxylates, carbonyls, and/or ether groups to the surface of the carbonaceous materials. The oxidizing agents and conditions used to merely remove amorphous carbon typically introduce less than 9 wt % oxygen to the surface of the carbon nanostructures.

Optionally, the purification process can also include additional heat treatment steps at temperatures and conditions that can convert residual amorphous carbon to graphite. In this optional step, residual carbon is more easily converted to a graphitic material since a substantial portion of the amorphous carbon has been removed and there is better heat transfer to the portion that remains. If desired, oxygen-containing functional groups can be introduced to the surface of the carbon nanospheres by treating the intermediate carbon nanomaterial with a severe oxidizing agent. Generally, the duration of the oxidative treatment will depend on the amount of amorphous carbon in the intermediate material (i.e., whether a prior purification step has been performed and if so, how much residual amorphous carbon remains), the strength of the oxidizing agent, and the desired amount of functional groups to be introduced. Typically, the rate of functionalization increases with decreasing residual amorphous carbon and increases with increasing oxidizing potential of the oxidizing agent. In one embodiment, the oxidative treatment is carried out for a period of time in a range from about 2 hours to about 48 hours. To facilitate oxidation, the oxidative treatment can be carried out using sonication.

Carbon nanomaterials manufactured using the foregoing methods can be particularly advantageous for use in the present invention due to their controlled size and shape. However, those skilled in the art will recognize that the present invention can be carried out using carbon nanospheres manufactured using different methods than the foregoing.

### V. EXAMPLES

The following examples provide formulas for making dispersed carbon nanomaterials containing carbon nanospheres according to the present invention.

### Example 1

Example 1 describes the preparation of an intermediate carbon nanomaterial having carbon nanospheres that are agglomerated into clusters with an average particle size greater than 1 micron as measured using dynamic light scattering.

(a) Preparation of Iron Solution (0.1 M)

A 0.1 M iron solution was prepared by using 84 g iron powder, 289 g of citric acid, and 15 L of water. The iron-containing mixture was mixed in a closed bottle on a shaker table for 3 days, with brief interruptions once or twice daily to purge the vapor space of the bottle with air gas before resuming mixing.

(b) Preparation and Curing of Precursor Mixture

916.6 g of resorcinol and 1350 g of formaldehyde (37% in water) were placed in a round bottom flask. The solution was stirred until resorcinol was fully dissolved. 15 L of the iron solution from step (a) was slowly added with stirring, and then 1025 ml of Ammonium hydroxide (28-30% in water) was added drop-wise with vigorous stirring, the pH of the resulted suspension was 10.26. The slurry was cured at 80˜90° C. (water bath) for 10 hours so as to polymerize the resorcinol and for maldehyde and form solid particles of polymerized carbonaceous material. The solid polymerized material formed in the precursor mixture were then collected using filtration and dried in an oven overnight.

(c) Carbonization

The polymerized material formed by curing the precursor mixture was placed in a crucible with a cover and transferred to a furnace. The carbonization process was carried out under ample nitrogen flow using the following temperature program: room temperature→1050° C. at a rate of 20° C./min→hold for 5 hrs at 1050° C.→room temperature. The carbonization step yielded an intermediate carbon material having carbon nanostructures, amorphous carbon, and iron.

(d) Purification to Remove Amorphous Carbon and Iron

The purification of the carbonized carbon product (i.e., the intermediate carbon material) was performed as follows: reflux carbonized product in 5M HNO₃ for ˜12 hrs→rinse with de-ionized (DI)-H₂O→treat with a mixture of KMnO₄+H₂SO₄+H₂O at a mole ratio of 1:0.01:0.003 (keep at ˜90° C. for ˜12 hrs)→rinse with DI-H₂O→treat with 4M HCl (keep at ˜90° C. for ˜12 hrs)→rinse with Di-H₂O→collect the product and dry in the oven at ˜100° C. for two days.

### Example 2

Example 2 describes a method for preparing dispersed carbon nanospheres using a dimeric cationic surfactant. 100 g of carbon nanospheres produced according to the method of Example 1 where suspended in 150 ml of ethanol and ultrasonicated for 1 hour. A surfactant mixture was prepared by mixing 20 g of cationic surfactant (C₁₂H₂₅N⁺(CH₃)₂(CH₂)2OOC(CH₂)₂COO(CH₂)2N⁺(CH₃)₂C₁₂H₂₅.2Br³) with 50 ml of ethanol. The surfactant mixture was reacted with the suspended carbon nanospheres by adding the surfactant mixture dropwise to the suspended carbon nanospheres and then stirring the mixture for 5 hours at 70° C. to yield dispersible carbon nanospheres. The dispersible carbon nanospheres were collected by filtration and washed with ample amounts of ethanol to remove the excess cationic surfactant. XPS was used to determine N% on the surface of CNS. About 2wt % of N was determined.

The method of Example 2 was repeated several times to produce Samples 1-4. The BET surface area, pore volume, and pore width of Samples 1-4 are provided in Table 1 below. Table 1 also includes the BET surface area, pore volume, and pore width of the untreated carbon nanospheres, which is identified as Sample 5.

| | BET surface area | Pore volume | Pore width |
|---|---|---|---|
| | before/after surface | before/after | before/after |
| Sample | treatment | surface treatment | surface treatment |
| 1 | 140.10/81.0 | 0.33/0.30 | 11.07/15.0 |
| 2 | 140.10/49.15 | 0.33/0.23 | 11.07/19.4 |
| 3 | 140.10/89.50 | 0.33/0.34 | 11.07/15.3 |
| 4 | 140.10/100.39 | 0.33/0.353 | 11.07/14.06 |

As shown in Table 1, the surface area of the carbon nanospheres manufactured according to the method of Example 2 resulted in substantially lower surface area, thereby indicating an increase in density. The sample was stable for months in a solvent, even after drying and resuspending the carbon nanospheres.

### Example 3

Example 3 describes a method for preparing dispersed carbon nanospheres using a cationic surfactant that includes an aromatic ring. 10 g of carbon nanospheres produced according to the method of Example 1 were suspended in 20 ml of ethanol and ultrasonicated for 1 hour. A surfactant mixture was prepared by mixing 5 g of hexadecylpyridine chloride monohydrate in 10 ml of ethanol. The surfactant mixture was reacted with the suspended carbon nanospheres by adding the surfactant mixture dropwise to the suspended carbon nanospheres and then stirring the mixture for 5 hours at 70° C. to yield dispersible carbon nanospheres. The dispersible carbon nanospheres were collected by filtration and washed with ample amounts of ethanol to remove the excess cationic surfactant.

The sample manufactured according to the method of Example 3 was tested for electrical conductivity. Surprisingly, the dispersible carbon nanospheres had a substantially higher conductivity compared to the undispersed carbon nanospheres. Specificially, the undispersed had a conductivity of 169.8±26.7 S/m and the dispersible carbon nanospheres had a conductivity of 315.3±7.6 S/m. Moreover, the increase in conductivity was found to be highly reproducible.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method for manufacturing dispersible carbon nanospheres, comprising:
(i) providing a carbon nanomaterial comprising a plurality of multi-walled, graphitic carbon nanospheres;
(ii) providing a surfactant solution that includes a cationic surfactant; and
(iii) mixing the carbon nanomaterial with the surfactant solution under conditions suitable for bonding the cationic surfactant to the graphitic carbon nanospheres to yield a dispersible carbon nanomaterial.

2. A method as in claim 1, wherein the carbon nanomaterial and the surfactant solution are sonicated.

3. A method as in claim 1, further comprising washing the dispersible carbon nanomaterial with a solvent suitable for removing unbounded cationic surfactant from the dispersible carbon nanomaterial

4. A method as in claim 1, wherein the cationic surfactant includes an aromatic ring.

5. A method as in claim 4, wherein the cationic surfactant includes a cationic group in the aromatic ring.

6. A method as in claim 4, wherein the cationic surfactant is selected from the group consisting of hexadecyl pyridine chloride, hexadecylpyridine, 1-(2-hydroxyethyl)pyridinium chloride, 1-(3-Cyanopropyl)pyridinium chloride, 1-Butyl-4-methylpyridinium chloride, 1-(1-(ethoxycarbonyl)tridecyl)pyridinium bromide.

7. A method as in claim 5, wherein the dispersible carbon nanomaterial has a conductivity of at least 50 S/m greater than the carbon nanomaterial provided in step (i).

8. A method as in claim 1, wherein the cationic surfactant includes at least two cationic groups.

9. A method as in claim 8, wherein the cationic groups are separated by a carbon chain of at least 2 carbons.

10. A method as in claim 8, wherein the cationic surfactant includes a compound selected from the group consisting of butane-1,4 bis(dodecyldimethyl ammonium chloride),

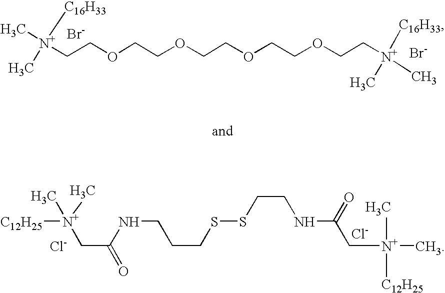

11. A method as in claim 1, wherein the surfactant solution includes a solvent selected from the group consisting of water, an alcohol, THF, DMF, or a combination thereof.

12. A method as in claim 1, wherein the carbon nanospheres are ultrasonicated for at least about 30 minutes.

13. A method as in claim 1, wherein the carbon nanospheres are manufactured by:
forming a precursor mixture comprising a carbon precursor and a plurality of templating nanoparticles and polymerizing the carbon precursor, the templating nanoparticles comprising a catalytic metal;
carbonizing the precursor mixture to form an intermediate carbon material comprising a plurality of carbon nanostructures, amorphous carbon, and optionally remaining catalytic metal; and
purifying the intermediate carbon material by removing at least a portion of the amorphous carbon and optionally a portion of any remaining catalytic metal, thereby yielding a carbon nanomaterial comprising a plurality of carbon nanostructures; and

14. A method as in claim 13, in which the templating nanoparticles are prepared by:
(a) reacting a plurality of precursor catalyst atoms with a plurality of organic dispersing agent molecules to form complexed catalyst atoms; and
(b) allowing or causing the complexed catalyst atoms to form the templating nanoparticles.

15. A dispersible carbon nanomaterial manufactured according to the method of claim 1.

16. A method for manufacturing dispersible carbon nanospheres, comprising:
(i) providing a carbon nanomaterial comprising a plurality of multi-walled, graphitic carbon nanospheres;
(ii) providing a surfactant solution that includes a cationic surfactant having an aromatic ring that includes a cationic group; and
(iii) mixing the carbon nanomaterial with the surfactant solution under conditions suitable for bonding the cationic surfactant to the graphitic carbon nanospheres to yield a dispersible carbon nanomaterial, wherein the mixing includes sonicating for at least about 30 minutes, the mixing yielding a dispersible carbon nanomaterial having a conductivity of at least about 200 S/m.

17. A method as in claim 16, wherein the cationic surfactant is selected from the group consisting of hexadecyl pyridine chloride, hexadecylpyridine, 1-(2-hydroxyethyl)pyridinium chloride, 1-(3-Cyanopropyl)pyridinium chloride, 1-Butyl-4-methylpyridinium chloride, 1-(1-(ethoxycarbonyl)tridecyl)pyridinium bromide.

18. A method as in claim 16, wherein the cationic surfactant includes a side chain group on the aromatic ring, the side chain having between 4 and 18 carbon atoms.

19. A method as in claim 16, wherein the dispersible carbon nanomaterial has a conductivity of at least about 300 S/m.

20. A method for manufacturing dispersible carbon nanospheres, comprising:
(i) providing a carbon nanomaterial comprising a plurality of multi-walled, graphitic carbon nanospheres;
(ii) providing a surfactant solution that includes a cationic surfactant having at least two cationic groups separated by a carbon chain; and
(iii) mixing the carbon nanomaterial with the surfactant solution under conditions suitable for bonding the cationic surfactant to the graphitic carbon nanospheres to yield a dispersible carbon nanomaterial, wherein the mixing includes sonicating for at least about 30 minutes, the mixing yielding a dispersible carbon nanomaterial having a density greater than about 0.4 g/ml.

21. A method as in claim 20, wherein the cationic surfactant is selected from the group consisting of butane-1,4 bis(dodecyldimethyl ammonium chloride), [chemical structure]
